(19) United States
(12) Patent Application Publication
(10) Pub. No.: US 2020/0289085 A1
(43) Pub. Date: Sep. 17, 2020

(54) FLEXIBLE TIP FOR INTRALUMINAL IMAGING DEVICE AND ASSOCIATED DEVICES, SYSTEMS, AND METHODS

(71) Applicant: KONINKLIJKE PHILIPS N.V., EINDHOVEN (NL)

(72) Inventors: Jeremy STIGALL, SAN DIEGO, CA (US); Maritess MINAS, SAN DIEGO, CA (US); Nathan Andrew WLLIAMS, SAN DIEGO, CA (US); Annamarie MENDOZA-CRUZ, SAN DIEGO, CA (US)

(21) Appl. No.: 16/768,170

(22) PCT Filed: Nov. 29, 2018
(86) PCT No.: PCT/EP2018/082951

§ 371 Date: May 29, 2020

Related U.S. Application Data
(60) Provisional application No. 62/595,744, filed on Dec. 7, 2017.

(51) Int. Cl. A61B 8/12; A61B 8/00; A61B 8/08

(52) U.S. Cl. CPC A61B 8/12; A61B 8/4236; A61B 8/0891; A61B 8/445

(57) ABSTRACT

An intraluminal imaging device is provided. The device includes a flexible elongate member configured to be inserted into a lumen of a patient, the flexible elongate member comprising a proximal portion and a distal portion. The device includes an ultrasound imaging assembly disposed at the distal portion and configured to obtain ultrasound imaging data while positioned within the lumen of the patient. The device includes a tip member disposed at the distal portion of the flexible elongate member, the tip member comprising a cavity adjacent to the ultrasound imaging assembly and configured to be filled with an adhesive to couple the tip member and the ultrasound imaging assembly. The tip member can include first material and a second material. The tip member can include linear outer diameter and varying wall thickness, and/or a varying outer diameter and a constant wall thickness.

### RELATED APPLICATION

This application claims the benefit of and priority to U.S. Provisional Application No. 62/595,744, filed Dec. 7, 2017, which is incorporated by reference in its entirety.

### TECHNICAL FIELD

The present disclosure relates generally to intraluminal ultrasound imaging and, in particular, to the structure of an intraluminal imaging device. For example, the intraluminal imaging device can include a flexible tip at the distal end of a flexible elongate member.

### BACKGROUND

Intravascular ultrasound (IVUS) imaging is widely used in interventional cardiology as a diagnostic tool for assessing a diseased vessel, such as an artery, within the human body to determine the need for treatment, to guide the intervention, and/or to assess its effectiveness. An IVUS device including one or more ultrasound transducers is passed into the vessel and guided to the area to be imaged. The transducers emit ultrasonic energy in order to create an image of the vessel of interest. Ultrasonic waves are partially reflected by discontinuities arising from tissue structures (such as the various layers of the vessel wall), red blood cells, and other features of interest. Echoes from the reflected waves are received by the transducer and passed along to an IVUS imaging system. The imaging system processes the received ultrasound echoes to produce a cross-sectional image of the vessel where the device is placed.

Solid-state (also known as synthetic-aperture) IVUS catheters are one of the two types of IVUS devices commonly used today, the other type being the rotational IVUS catheter. Solid-state IVUS catheters carry a scanner assembly that includes an array of ultrasound transducers distributed around its circumference along with one or more integrated circuit controller chips mounted adjacent to the transducer array. The controllers select individual transducer elements (or groups of elements) for transmitting an ultrasound pulse and for receiving the ultrasound echo signal. By stepping through a sequence of transmit-receive pairs, the solid-state IVUS system can synthesize the effect of a mechanically scanned ultrasound transducer but without moving parts (hence the solid-state designation). Since there is no rotating mechanical element, the transducer array can be placed in direct contact with the blood and vessel tissue with minimal risk of vessel trauma. Furthermore, because there is no rotating element, the electrical interface is simplified. The solid-state scanner can be wired directly to the imaging system with a simple electrical cable and a standard detachable electrical connector, rather than the complex rotating electrical interface required for a rotational IVUS device.

Manufacturing an intravascular imaging device that can efficiently traverse physiology within the human body is challenging. In that regard, components at the distal portion of the imaging device can be assembled in a manner that excessively enlarges an outer diameter, which makes navigation through smaller diameter vessels difficult. Ensuring robust mechanical coupling between components can also be challenging.

### SUMMARY

Intraluminal imaging devices are inserted into the human body to obtain information regarding the condition of various anatomies therein. For example, the intraluminal imaging device, such as an intravascular ultrasound (IVUS) device, can be introduced into to the body through a blood vessel and then guided to an anatomical area of interest. It is common for the intraluminal imaging device to encounter various obstructions while traveling within the body. In response to this, a front end of the intraluminal imaging device has been equipped with a tip member to facilitate the navigation of the intraluminal imaging device through the body. An outer profile of the tip member may be conical in shape and decrease in diameter from a leading front end of the tip member to a back end. The front end of the tip member may be formed using a material that is more flexible than the material used to form the back end of the tip. The tip member may be connected to the intraluminal imaging device by the application of an adhesive around the outer profile of each. To minimize impact the adhesive has on the outer profile of the tip member and the intraluminal imaging device, a cavity is formed in the proximal end of the tip member to receive the adhesive. The cavity functions to provide both a connection and a seal between the intraluminal imaging device and the tip member. The profile and flexible nature of the tip member assist the intraluminal imaging device in navigating obstructions while being guided through the body. Embodiments described herein advantageously minimize the outer diameter of the imaging assembly while achieving strong and efficient assembly and operation.

In an exemplary aspect, an intraluminal imaging device is provided. The device includes a flexible elongate member configured to be inserted into a lumen of a patient, the flexible elongate member comprising a proximal portion and a distal portion; an ultrasound imaging assembly disposed at the distal portion and configured to obtain ultrasound imaging data while positioned within the lumen of the patient; and a tip member disposed at the distal portion of the flexible elongate member, the tip member comprising a cavity adjacent to the ultrasound imaging assembly and configured to be filled with an adhesive to couple the tip member and the ultrasound imaging assembly.

In some aspects, the cavity comprises a junction region at a proximal portion of the tip member, and the cavity comprises a smaller outer diameter relative to the proximal portion of the tip member. In some aspects, the cavity comprises a linear outer diameter. In some aspects, the cavity further comprises a sloped outer diameter. In some aspects, a distal portion of the tip member comprises a crossing region configured to cross an occlusion of the lumen, wherein an outer diameter of the crossing region decreases along a longitudinal axis of the flexible elongate member. In some aspects, the crossing region of the tip member comprises a linear outer diameter. In some aspects, the crossing region of the tip member comprises a curvilinear outer diameter. In some aspects, a distal end of the tip member is shaped to facilitate crossing the occlusion. In some aspects, the distal end of the tip member comprises a linear outer diameter. In some aspects, the distal end of the tip member comprises a curvilinear outer diameter. In some aspects, the distal end of the tip member comprises a reinforcing apparatus. In some aspects, the reinforcing apparatus comprises a first color and the tip member comprises a second color different than the first color. In some aspects, the proximal portion of the tip member comprises a first material and the distal portion of the tip member comprises a second material. In some aspects, the tip member comprises an inner diameter associated with a lumen extending therethrough, wherein the inner diameter comprises an engagement feature configured to contact at least portion of the ultrasound imaging assembly disposed within the lumen.

In an exemplary aspect, an intraluminal imaging device is provided. The device includes a flexible elongate member configured to be inserted into a lumen of a patient, the flexible elongate member comprising a proximal portion and a distal portion; an ultrasound imaging assembly disposed at the distal portion and configured to obtain ultrasound imaging data while positioned within the lumen of the patient; and a tip member at the distal portion of the flexible elongate member and comprising a first material at a distal portion of the tip member and a second material at a proximal portion of the tip member.

In some aspects, the first material is less rigid than the second material such that the distal portion of the tip member is more flexible than the proximal portion of the tip member. In some aspects, the device further includes a transition region between the proximal portion and the distal portion, the transition region comprised of the first material and the second material.

In an exemplary aspect, an intraluminal imaging device is provided. The device includes a flexible elongate member configured to be inserted into a lumen of a patient, the flexible elongate member comprising a proximal portion and a distal portion; an ultrasound imaging assembly disposed at the distal portion and configured to obtain ultrasound imaging data while positioned within the lumen of the patient; and a tip member at the distal portion of the flexible elongate member and comprising a proximal portion and a distal portion, wherein the proximal portion of the tip member comprises linear outer diameter and varying wall thickness, and the distal portion of the tip member comprises a varying outer diameter and a constant wall thickness.

In some aspects, the wall thickness of the proximal portion of the tip member in is greater than the wall thickness of the distal portion of the tip member.

Additional aspects, features, and advantages of the present disclosure will become apparent from the following detailed description.

### BRIEF DESCRIPTION OF THE DRAWINGS

Illustrative embodiments of the present disclosure will be described with reference to the accompanying drawings, of which:

FIG. 1 is a diagrammatic schematic view of an imaging system, according to aspects of the present disclosure.

FIG. 2 is a diagrammatic top view of a scanner assembly in a flat configuration, according to aspects of the present disclosure.

FIG. 3 is a diagrammatic side view of a scanner assembly in a rolled configuration around a support member, according to aspects of the present disclosure.

FIG. 4 is a diagrammatic cross sectional side view of a distal portion of an intravascular device, according to aspects of the present disclosure.

FIG. 5a is a diagrammatic cross sectional side view of a tip member joint of an intraluminal device, according to aspects of the present disclosure.

FIG. 5b is a diagrammatic cross sectional side view of a tip member joint of an intraluminal device, according to aspects of the present disclosure.

FIG. 5c is a diagrammatic cross sectional side view of a tip member of an intraluminal device, according to aspects of the present disclosure.

FIG. 6a is a perspective view illustration of a tip member of an intraluminal device, according to aspects of the present disclosure.

FIG. 6b is a diagrammatic cross sectional side view of a tip member and imaging assembly, according to aspects of the present disclosure.

FIG. 7 is a diagrammatic cross sectional side view of a tip member of an intraluminal device, according to aspects of the present disclosure.

FIG. 8 is a diagrammatic cross sectional side view of a tip member of an intraluminal device, according to aspects of the present disclosure.

FIG. 9 is a side illustration of a tip member with a ramp type crossing profile, according to aspects of the present disclosure.

FIG. 10 is a side view illustration of a tip member with a slope type crossing profile, according to aspects of the present disclosure.

FIG. 11 is a side view illustration of a tip member with a step type crossing profile, according to aspects of the present disclosure.

FIG. 12 is a diagrammatic cross sectional side view illustration of a tip member with a bevel distal end, according to aspects of the present disclosure.

FIG. 13 is a diagrammatic cross sectional side view illustration of a tip member with a radial distal end, according to aspects of the present disclosure.

FIG. 14 is a diagrammatic cross sectional side view illustration of a tip member with a reinforced radial distal end, according to aspects of the present disclosure.

### DETAILED DESCRIPTION

For the purposes of promoting an understanding of the principles of the present disclosure, reference will now be made to the embodiments illustrated in the drawings, and specific language will be used to describe the same. It is nevertheless understood that no limitation to the scope of the disclosure is intended. Any alterations and further modifications to the described devices, systems, and methods, and any further application of the principles of the present disclosure are fully contemplated and included within the present disclosure as would normally occur to one skilled in the art to which the disclosure relates. For example, while the focusing system is described in terms of cardiovascular imaging, it is understood that it is not intended to be limited to this application. The system is equally well suited to any application requiring imaging within a confined cavity. In particular, it is fully contemplated that the features, components, and/or steps described with respect to one embodiment may be combined with the features, components, and/or steps described with respect to other embodiments of the present disclosure. For the sake of brevity, however, the numerous iterations of these combinations will not be described separately.

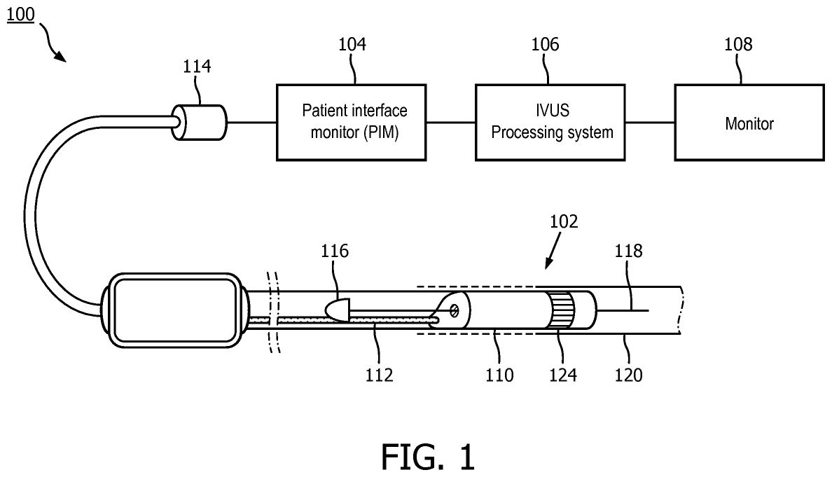

FIG. 1 is a diagrammatic schematic view of an intraluminal imaging system 100, according to aspects of the present disclosure. For example, the system 100 can be an intraluminal ultrasound imaging system or intravascular ultrasound (IVUS) imaging system. The imaging system 100 may include an intraluminal ultrasound imaging device 102 such as a catheter, guide wire, or guide catheter, a patient interface module (PIM) 104, a processing system or console 106, and a monitor 108.

At a high level, the IVUS device 102 emits ultrasonic energy from a transducer array 124 included in scanner assembly 110 mounted near a distal end of the catheter device. The ultrasonic energy is reflected by tissue structures in the medium, such as a vessel 120, surrounding the scanner assembly 110, and the ultrasound echo signals are received by the transducer array 124. The PIM 104 transfers the received echo signals to the console or computer 106 where the ultrasound image (including the flow information) is reconstructed and displayed on the monitor 108. The console or computer 106 can include a processor and a memory. The computer or computing device 106 can be operable to facilitate the features of the imaging system 100 described herein. For example, the processor can execute computer readable instructions stored on the non-transitory tangible computer readable medium.

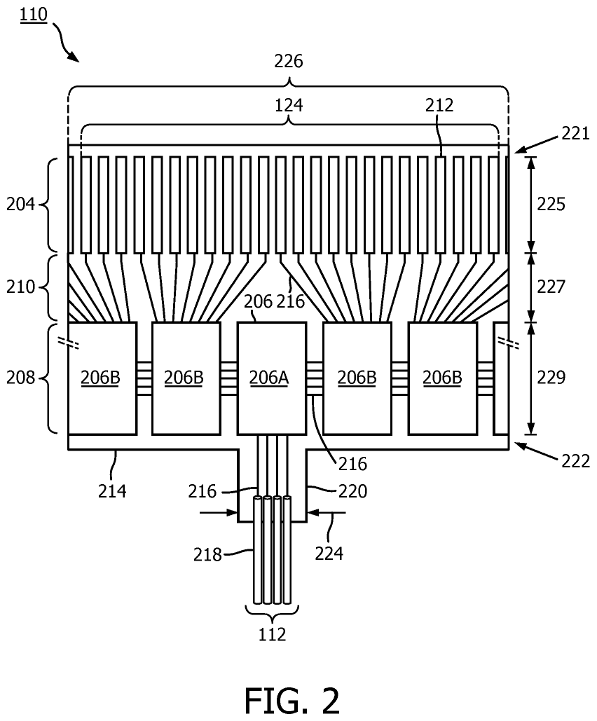

The PIM 104 facilitates communication of signals between the console 106 and the scanner assembly 110 included in the IVUS device 102. This communication includes the steps of: (1) providing commands to integrated circuit controller chip(s) 206A, 206B, illustrated in FIG. 2, included in the scanner assembly 110 to select the particular transducer array element(s) to be used for transmit and receive, (2) providing the transmit trigger signals to the integrated circuit controller chip(s) 206A, 206B included in the scanner assembly 110 to activate the transmitter circuitry to generate an electrical pulse to excite the selected transducer array element(s), and/or (3) accepting amplified echo signals received from the selected transducer array element(s) via amplifiers included on the integrated circuit controller chip(s)126 of the scanner assembly 110. In some embodiments, the PIM 104 performs preliminary processing of the echo data prior to relaying the data to the console 106. In examples of such embodiments, the PIM 104 performs amplification, filtering, and/or aggregating of the data. In an embodiment, the PIM 104 also supplies high- and low-voltage DC power to support operation of the device 102 including circuitry within the scanner assembly 110.

The console 106 receives the echo data from the scanner assembly 110 by way of the PIM 104 and processes the data to reconstruct an image of the tissue structures in the medium surrounding the scanner assembly 110. For the example, the device 102 can be sized and shaped, structurally arranged, and/or otherwise configured to be positioned with a body lumen 120 of the patient. For example, the body lumen 120 can be a vessel in some embodiments. The console 106 outputs image data such that an image of the body lumen 120, such as a cross-sectional image of the vessel 120, is displayed on the monitor 108. Lumen 120 may represent fluid filled or surrounded structures, both natural and man-made. The lumen 120 may be within a body of a patient. The lumen 120 may be a blood vessel, such as an artery or a vein of a patient's vascular system, including cardiac vasculature, peripheral vasculature, neural vasculature, renal vasculature, and/or or any other suitable lumen inside the body. For example, the device 102 may be used to examine any number of anatomical locations and tissue types, including without limitation, organs including the liver, heart, kidneys, gall bladder, pancreas, lungs; ducts; intestines; nervous system structures including the brain, dural sac, spinal cord and peripheral nerves; the urinary tract; as well as valves within the blood, chambers or other parts of the heart, and/or other systems of the body. In addition to natural structures, the device 102 may be may be used to examine man-made structures such as, but without limitation, heart valves, stents, shunts, filters and other devices.

In various embodiments, the intraluminal imaging device 102 and/or the imaging assembly 110 can obtain imaging data associated with intravascular ultrasound (IVUS) imaging, forward looking intravascular ultrasound (FL-IVUS) imaging, intravascular photoacoustic (IVPA) imaging, intracardiac echocardiography (ICE), forward-looking ICE (FLICE), transesophageal echocardiography (TEE), optical coherence tomography (OCT), and/or other suitable imaging modalities. The system 100 and/or the device 102 may also be configured to obtain physiologic data associated with pressure, flow, temperature, a fractional flow reserve (FFR) determination, a functional measurement determination, a coronary flow reserve (CFR) determination, radiographic imaging, angiographic imaging, fluoroscopic imaging, computed tomography (CT), magnetic resonance imaging (MRI), intravascular palpography, and/or other types of physiologic data.

In some embodiments, the IVUS device includes some features similar to traditional solid-state IVUS catheters, such as the EagleEye® catheter available from Volcano Corporation and those disclosed in U.S. Pat. No. 7,846,101 hereby incorporated by reference in its entirety. For example, the IVUS device 102 includes the scanner assembly 110 near a distal end of the device 102 and a transmission line bundle 112 extending along the longitudinal body of the device 102. The transmission line bundle or cable 112 can include a plurality of conductors, including one, two, three, four, five, six, seven, or more conductors 218 (FIG. 2). It is understood that any suitable gauge wire can be used for the conductors 218. In an embodiment, the cable 112 can include a four-conductor transmission line arrangement with, e.g., 41 AWG gauge wires. In an embodiment, the cable 112 can include a seven-conductor transmission line arrangement utilizing, e.g., 44 AWG gauge wires. In some embodiments, 43 AWG gauge wires can be used.

The transmission line bundle 112 terminates in a PIM connector 114 at a proximal end of the device 102. The PIM connector 114 electrically couples the transmission line bundle 112 to the PIM 104 and physically couples the IVUS device 102 to the PIM 104. In an embodiment, the IVUS device 102 further includes a guide wire exit port 116. Accordingly, in some instances the IVUS device is a rapid-exchange catheter. The guide wire exit port 116 allows a guide wire 118 to be inserted towards the distal end in order to direct the device 102 through the vessel 120.

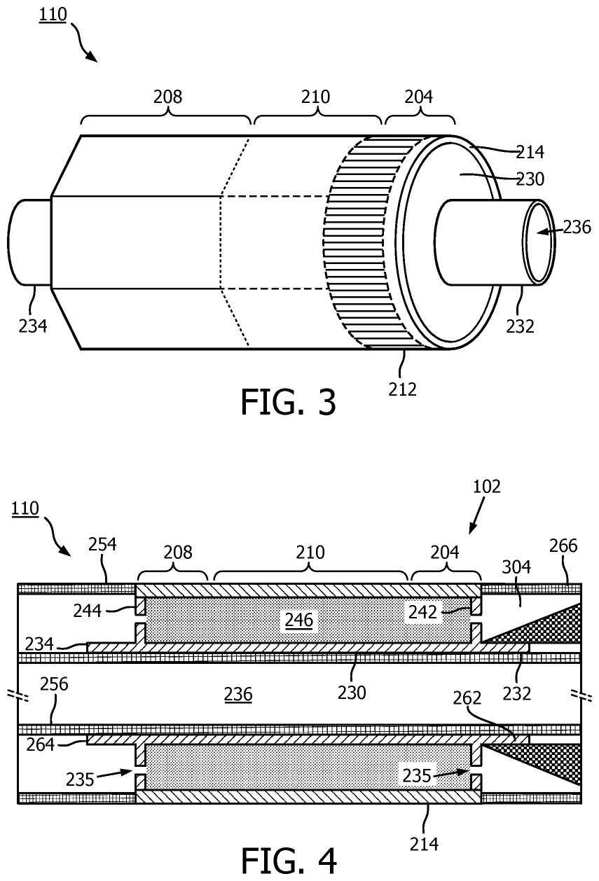

FIG. 2 is a top view of a portion of an ultrasound scanner assembly 110 according to an embodiment of the present disclosure. The assembly 110 includes a transducer array 124 formed in a transducer region 204 and transducer control logic dies 206 (including dies 206A and 206B) formed in a control region 208, with a transition region 210 disposed therebetween. The transducer control logic dies 206 and the transducers 212 are mounted on a flex circuit 214 that is shown in a flat configuration in FIG. 2. FIG. 3 illustrates a rolled configuration of the flex circuit 214. The transducer array 202 is a non-limiting example of a medical sensor element and/or a medical sensor element array. The transducer control logic dies 206 is a non-limiting example of a control circuit. The transducer region 204 is disposed adjacent a distal portion 221 of the flex circuit 214. The control region 208 is disposed adjacent the proximal portion 222 of the flex circuit 214. The transition region 210 is disposed between the control region 208 and the transducer region 204. Dimensions of the transducer region 204, the control region 208, and the transition region 210 (e.g., lengths 225, 227, 229) can vary in different embodiments. In some embodiments, the lengths 225, 227, 229 can be substantially similar or a length 227 of the transition region 210 can be greater than lengths 225, 229 of the transducer region and controller region, respectively. While the imaging assembly 110 is described as including a flex circuit, it is understood that the transducers and/or controllers may be arranged to form the imaging assembly 110 in other configurations, including those omitting a flex circuit.

The transducer array 124 may include any number and type of ultrasound transducers 212, although for clarity only a limited number of ultrasound transducers are illustrated in FIG. 2. In an embodiment, the transducer array 124 includes 64 individual ultrasound transducers 212. In a further embodiment, the transducer array 124 includes 32 ultrasound transducers 212. Other numbers are both contemplated and provided for. With respect to the types of transducers, in an embodiment, the ultrasound transducers 124 are piezoelectric micromachined ultrasound transducers (PMUTs) fabricated on a microelectromechanical system (MEMS) substrate using a polymer piezoelectric material, for example as disclosed in U.S. Pat. No. 6,641,540, which is hereby incorporated by reference in its entirety. In alternate embodiments, the transducer array includes piezoelectric zirconate transducers (PZT) transducers such as bulk PZT transducers, capacitive micromachined ultrasound transducers (cMUTs), single crystal piezoelectric materials, other suitable ultrasound transmitters and receivers, and/or combinations thereof.

The scanner assembly 110 may include various transducer control logic, which in the illustrated embodiment is divided into discrete control logic dies 206. In various examples, the control logic of the scanner assembly 110 performs: decoding control signals sent by the PIM 104 across the cable 112, driving one or more transducers 212 to emit an ultrasonic signal, selecting one or more transducers 212 to receive a reflected echo of the ultrasonic signal, amplifying a signal representing the received echo, and/or transmitting the signal to the PIM across the cable 112. In the illustrated embodiment, a scanner assembly 110 having 64 ultrasound transducers 212 divides the control logic across nine control logic dies 206, of which five are shown in FIG. 2. Designs incorporating other numbers of control logic dies 206 including 8, 9, 16, 17 and more are utilized in other embodiments. In general, the control logic dies 206 are characterized by the number of transducers they are capable of driving, and exemplary control logic dies 206 drive 4, 8, and/or 16 transducers.

The control logic dies are not necessarily homogenous. In some embodiments, a single controller is designated a master control logic die 206A and contains the communication interface for the cable 112. Accordingly, the master control circuit may include control logic that decodes control signals received over the cable 112, transmits control responses over the cable 112, amplifies echo signals, and/or transmits the echo signals over the cable 112. The remaining controllers are slave controllers 206B. The slave controllers 206B may include control logic that drives a transducer 212 to emit an ultrasonic signal and selects a transducer 212 to receive an echo. In the depicted embodiment, the master controller 206A does not directly control any transducers 212. In other embodiments, the master controller 206A drives the same number of transducers 212 as the slave controllers 206B or drives a reduced set of transducers 212 as compared to the slave controllers 206B. In an exemplary embodiment, a single master controller 206A and eight slave controllers 206B are provided with eight transducers assigned to each slave controller 206B.

The flex circuit 214, on which the transducer control logic dies 206 and the transducers 212 are mounted, provides structural support and interconnects for electrical coupling. The flex circuit 214 may be constructed to include a film layer of a flexible polyimide material such as KAPTON™ (trademark of DuPont). Other suitable materials include polyester films, polyimide films, polyethylene napthalate films, or polyetherimide films, other flexible printed semiconductor substrates as well as products such as Upilex® (registered trademark of Ube Industries) and TEFLON® (registered trademark of E.I. du Pont). In the flat configuration illustrated in FIG. 2, the flex circuit 214 has a generally rectangular shape. As shown and described herein, the flex circuit 214 is configured to be wrapped around a support member 230 (FIG. 3) to form a cylindrical toroid in some instances. Therefore, the thickness of the film layer of the flex circuit 214 is generally related to the degree of curvature in the final assembled scanner assembly 110. In some embodiments, the film layer is between 5 μm and 100 μm, with some particular embodiments being between 12.7 μm and 25.1 μm.

To electrically interconnect the control logic dies 206 and the transducers 212, in an embodiment, the flex circuit 214 further includes conductive traces 216 formed on the film layer that carry signals between the control logic dies 206 and the transducers 212. In particular, the conductive traces 216 providing communication between the control logic dies 206 and the transducers 212 extend along the flex circuit 214 within the transition region 210. In some instances, the conductive traces 216 can also facilitate electrical communication between the master controller 206A and the slave controllers 206B. The conductive traces 216 can also provide a set of conductive pads that contact the conductors 218 of cable 112 when the conductors 218 of the cable 112 are mechanically and electrically coupled to the flex circuit 214. Suitable materials for the conductive traces 216 include copper, gold, aluminum, silver, tantalum, nickel, and tin, and may be deposited on the flex circuit 214 by processes such as sputtering, plating, and etching. In an embodiment, the flex circuit 214 includes a chromium adhesion layer. The width and thickness of the conductive traces 216 are selected to provide proper conductivity and resilience when the flex circuit 214 is rolled. In that regard, an exemplary range for the thickness of a conductive trace 216 and/or conductive pad is between 10-50 μm. For example, in an embodiment, 20 μm conductive traces 216 are separated by 20 μm of space. The width of a conductive trace 216 on the flex circuit 214 may be further determined by the width of the conductor 218 to be coupled to the trace/pad.

The flex circuit 214 can include a conductor interface 220 in some embodiments. The conductor interface 220 can be a location of the flex circuit 214 where the conductors 218 of the cable 114 are coupled to the flex circuit 214. For example, the bare conductors of the cable 114 are electrically coupled to the flex circuit 214 at the conductor interface 220. The conductor interface 220 can be tab extending from the main body of flex circuit 214. In that regard, the main body of the flex circuit 214 can refer collectively to the transducer region 204, controller region 208, and the transition region 210. In the illustrated embodiment, the conductor interface 220 extends from the proximal portion 222 of the flex circuit 214. In other embodiments, the conductor interface 220 is positioned at other parts of the flex circuit 214, such as the distal portion 220, or the flex circuit 214 omits the conductor interface 220. A value of a dimension of the tab or conductor interface 220, such as a width 224, can be less than the value of a dimension of the main body of the flex circuit 214, such as a width 226. In some embodiments, the substrate forming the conductor interface 220 is made of the same material(s) and/or is similarly flexible as the flex circuit 214. In other embodiments, the conductor interface 220 is made of different materials and/or is comparatively more rigid than the flex circuit 214. For example, the conductor interface 220 can be made of a plastic, thermoplastic, polymer, hard polymer, etc., including polyoxymethylene (e.g., DELRIN®), polyether ether ketone (PEEK), nylon, and/or other suitable materials. As described in greater detail herein, the support member 230, the flex circuit 214, the conductor interface 220 and/or the conductor(s) 218 can be variously configured to facilitate efficient manufacturing and operation of the scanner assembly 110.

In some instances, the scanner assembly 110 is transitioned from a flat configuration (FIG. 2) to a rolled or more cylindrical configuration (FIGS. 3 and 4). For example, in some embodiments, techniques are utilized as disclosed in one or more of U.S. Pat. No. 6,776,763, titled “ULTRASONIC TRANSDUCER ARRAY AND METHOD OF MANUFACTURING THE SAME” and U.S. Pat. No. 7,226,417, titled “HIGH RESOLUTION INTRAVASCULAR ULTRASOUND TRANSDUCER ASSEMBLY HAVING A FLEXIBLE SUBSTRATE,” each of which is hereby incorporated by reference in its entirety.

As shown in FIGS. 3 and 4, the flex circuit 214 is positioned around the support member 230 in the rolled configuration. FIG. 3 is a diagrammatic side view with the flex circuit 214 in the rolled configuration around the support member 230, according to aspects of the present disclosure. FIG. 4 is a diagrammatic cross-sectional side view of a distal portion of the intravascular device 110, including the flex circuit 214 the support member 230 and a tip member 304, according to aspects of the present disclosure.

The support member 230 can be referenced as a unibody in some instances. The support member 230 can be composed of a metallic material, such as stainless steel, or non-metallic material, such as a plastic or polymer as described in U.S. Provisional Application No. 61/985,220, “Pre-Doped Solid Substrate for Intravascular Devices,” filed Apr. 28, 2014, the entirety of which is hereby incorporated by reference herein. The support member 230 can be ferrule having a distal portion 262 and a proximal portion 264. The support member 230 can define a lumen 236 extending longitudinally therethrough. The lumen 236 is in communication with the exit port 116 and is sized and shaped to receive the guide wire 118 (FIG. 1). The support member 230 can be manufactured accordingly to any suitable process. For example, the support member 230 can be machined, such as by removing material from a blank to shape the support member 230, or molded, such as by an injection molding process. In some embodiments, the support member 230 may be integrally formed as a unitary structure, while in other embodiments the support member 230 may be formed of different components, such as a ferrule and stands 242, 244, that are fixedly coupled to one another.

Stands 242, 244 that extend vertically are provided at the distal and proximal portions 262, 264, respectively, of the support member 230. The stands 242, 244 elevate and support the distal and proximal portions of the flex circuit 214. In that regard, portions of the flex circuit 214, such as the transducer portion 204, can be spaced from a central body portion of the support member 230 extending between the stands 242, 244. The stands 242, 244 can have the same outer diameter or different outer diameters. For example, the distal stand 242 can have a larger or smaller outer diameter than the proximal stand 244. To improve acoustic performance, any cavities between the flex circuit 214 and the surface of the support member 230 are filled with a backing material 246. The liquid backing material 246 can be introduced between the flex circuit 214 and the support member 230 via passageways 235 in the stands 242, 244. In some embodiments, suction can be applied via the passageways 235 of one of the stands 242, 244, while the liquid backing material 246 is fed between the flex circuit 214 and the support member 230 via the passageways 235 of the other of the stands 242, 244. The backing material can be cured to allow it to solidify and set. In various embodiments, the support member 230 includes more than two stands 242, 244, only one of the stands 242, 244, or neither of the stands. In that regard the support member 230 can have an increased diameter distal portion 262 and/or increased diameter proximal portion 264 that is sized and shaped to elevate and support the distal and/or proximal portions of the flex circuit 214.

The support member 230 can be substantially cylindrical in some embodiments. Other shapes of the support member 230 are also contemplated including geometrical, non-geometrical, symmetrical, non-symmetrical, cross-sectional profiles. Different portions the support member 230 can be variously shaped in other embodiments. For example, the proximal portion 264 can have a larger outer diameter than the outer diameters of the distal portion 262 or a central portion extending between the distal and proximal portions 262, 264. In some embodiments, an inner diameter of the support member 230 (e.g., the diameter of the lumen 236) can correspondingly increase or decrease as the outer diameter changes. In other embodiments, the inner diameter of the support member 230 remains the same despite variations in the outer diameter.

A proximal inner member 256 and a proximal outer member 254 are coupled to the proximal portion 264 of the support member 230. The proximal inner member 256 and/or the proximal outer member 254 can be flexible elongate member that extend from proximal portion of the intravascular 102, such as the proximal connector 114, to the imaging assembly 110. For example, the proximal inner member 256 can be received within a proximal flange 234. The proximal outer member 254 abuts and is in contact with the flex circuit 214. A tip member 304 is coupled to the distal portion 262 of the support member 230. As discussed further herein, the tip member 304 can be a flexible component that defines a distal most portion of the intravascular device 102. For example, the tip member 304 is positioned around the distal flange 232. The tip member 304 can abut and be in contact with the flex circuit 214 and the stand 242. The tip member 304 can be the distal-most component of the intravascular device 102. The tip member 304 functions to facilitate the translation of the intraluminal device 300, through any number of anatomies encountered in a patient, including but not limited to lesions and blood vessels with short radii.

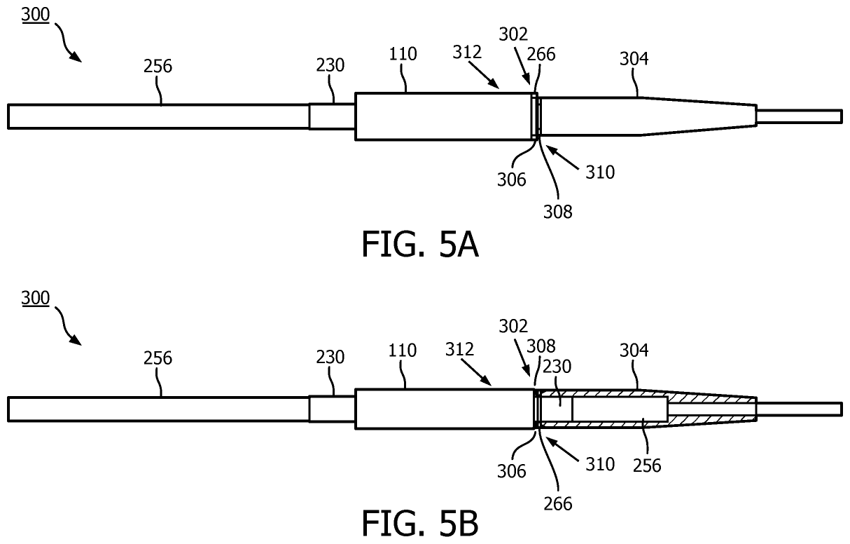

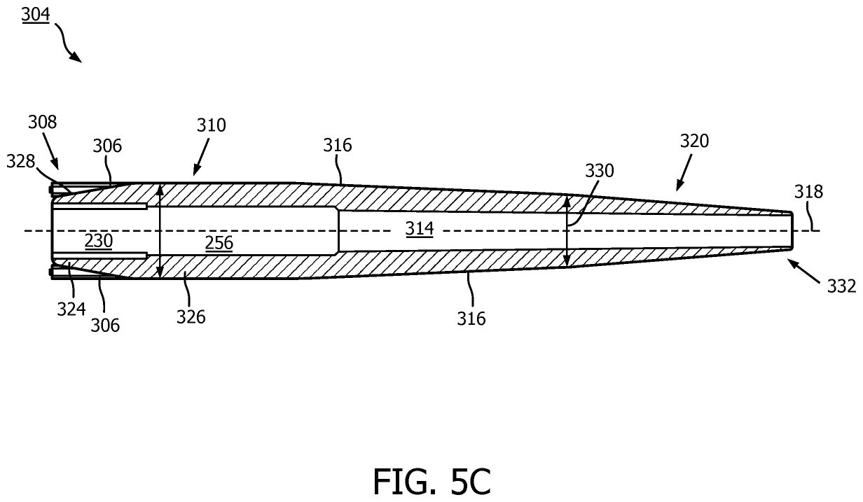

FIGS. 5a and 5b illustrate an embodiment of an intraluminal device 300, including a joint 302 which facilitates the connection of the imaging assembly 110, which in certain embodiments is a scanner assembly, and the tip member 304. FIG. 5a is a side view illustration of the imaging assembly 110 and the tip member 304 joint 302. FIG. 5b is a cross-sectional side view illustration of the imaging assembly 110 and the tip member 304 joint 302. For clarity, the proximal portion of the intraluminal device 300 is shown the left side of FIGS. 5a and 5b, and more distal portions are shown on the right side.

The intraluminal device 300 can be similar to the intravascular device 102 in some aspects. With reference to FIGS. 5a and 5b, the imaging assembly 110 and the tip member 304 joint 302 may include an adhesive 306 disposed at a junction region 308 positioned between a proximal portion 310 of the tip member 304 and the distal end 312 of the imaging assembly 110. The adhesive 306 functions to mechanically connect the imaging assembly 110 and the tip member 304. Further, the adhesive 306 functions to provide a hermetic seal between the tip member 304 and the distal end 312 of the imaging assembly 110. As discussed further herein, the junction region 308 is configured to receive the adhesive 306 while limiting the overall diameter of the tip member 304 and the joint 302. It is anticipated that one or more adhesives 306 may be disposed in the junction region 308. The adhesive 306 may be disposed within the junction region 308 such that a limited amount of adhesive 306 overlaps the imaging assembly 110 and the proximal portion 310 of the tip member 304. FIG. 5b provides an illustration of the support member 230 and the inner member 256 extending through the junction region 308 and into the proximal portion 310 of the tip member 304.

Turning now to FIG. 5c, a cross-sectional view of the tip member 304 is presented. The tip member 304 may include a lumen 314 extending between the walls 316 of the tip member 304 along a longitudinal axis 318 between the junction region 308, the proximal portion 310 and a distal portion 320. It will be appreciated that the respective lengths and geometrical profiles of the junction region 308, the proximal portion 310 and the distal portion 320 may vary in accordance with the functional objective of the tip member 304 as discussed further herein. FIG. 5c depicts the walls 316 sloping in a linear fashion from the proximal portion 310 to the distal portion 320. However as described further herein, the walls 316 may also slope in a curvilinear fashion. The walls 316 and the lumen 314 may define an inner diameter 322 of the tip member 304. An engagement feature 324 may be positioned along the inner diameter 322 to secure the support member 230 within the proximal portion 310. It is anticipated that the engagement feature 324 may include any number of securing mechanisms or methods as known in the art, such as, but not limited to surface roughening, grooves, threads to secure the support member 230 to the inner diameter 322 of the tip member 304.

The tip member 304 may also include a crossing region 326, which may be defined as the area of the tip member 304 containing the largest outer diameter of the tip member 304 profile and is generally located in the proximal portion 310 or the junction region 308.

The junction region 308 is disposed between the proximal portion 310 of the tip member 304 and the imaging assembly 110 within the joint 302. The junction region 308 includes a cavity 328 for receiving the adhesive 306 used to facilitate a mechanical connection between the imaging assembly 110 and the tip member 304. The cavity 328 may be configured to receive the adhesive 306 for the mechanical connection, while at the same time functioning to minimize the crossing region 326 of the tip member 304. It is anticipated however, that the addition of adhesive 306 to the junction region 308 may increase the overall diameter of the tip member 304 becoming the de facto location of the crossing region 326. This may particularly be the case where it is desired to create an adhesive 306 overlap in the joint 302 between the imaging assembly 110 and the tip member 304 as previously discussed. As shown in FIG. 5c, the cavity 328 of the junction region 308 may be defined by a linear slope of the wall 316 that extends away from the proximal portion 310 towards the imaging assembly 110, which forms an annular triangular cross section. However, as discussed further herein the cavity 328 may be defined by any number of geometries which facilitate minimizing the crossing region 326 of the tip member 304.

With continued reference to FIG. 5c, the wall 316 is also shown linearly sloping away from the proximal portion 310 of the tip member 304 towards the distal portion 320 of the tip member. In this configuration, the outer diameter 330 of the tip member 304 gradually decreases along the longitudinal axis 318 from the proximal portion 310 to the distal portion 320. At the most distal position of the distal portion 320 is the distal end 332, which as discussed further herein, is the first point of contact between the tip member 304 of the intraluminal device 300 and any obstruction along the path of the intraluminal device 300.

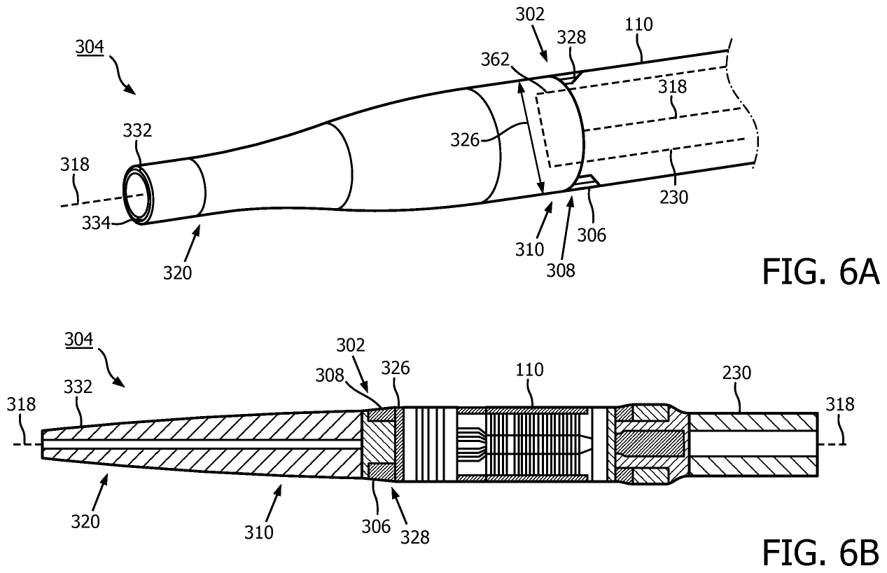

FIGS. 6a and 6b illustrate an enlarged perspective and diagrammatic cross sectional view of the imaging assembly 110 and the tip member 304 joint 302, respectively. FIG. 6a illustrates the support member 230 of the imaging assembly 110 extending through the junction region 308 of the tip member 304 to the proximal portion 310. The cavity 328 is shown an in annular configuration with a trapezoidal cross-section. In contrast to the linear slopes depicted in FIGS. 5a-5c, in FIG. 6a the tip member 304 is shown with a partial curvilinear profile that decreases along the longitudinal axis 318 from the proximal portion 310 to the distal portion 320. The distal end 332 of the distal portion 320 contains a reinforcing apparatus 334, which in certain embodiments is a reinforcement ring, positioned between the inner diameter 322 and the lumen 314 of the tip member 304. As discussed further herein, the reinforcement apparatus 334 functions to provide rigidity to the distal portion 320 tip member 304. This rigidity will prevent the deformation of the tip member 304 upon encountering a relatively rigid obstruction along the along the path of the intraluminal device 300.

FIG. 6b depicts a configuration of the tip member 304 with a linear profile that decreases along the longitudinal axis 318 from the proximal portion 310 to the distal portion 320 similar as to illustrated in FIGS. 5a-5c. This configuration however, illustrates an annular cavity 328 containing adhesive 306, which has a rectangular cross section in contrast with the triangular and trapezoidal cross sections previously described. It will be appreciated that tip member 304 may be comprised of any number of combinations of geometrical shaped profiles and cavity 328 cross sections.

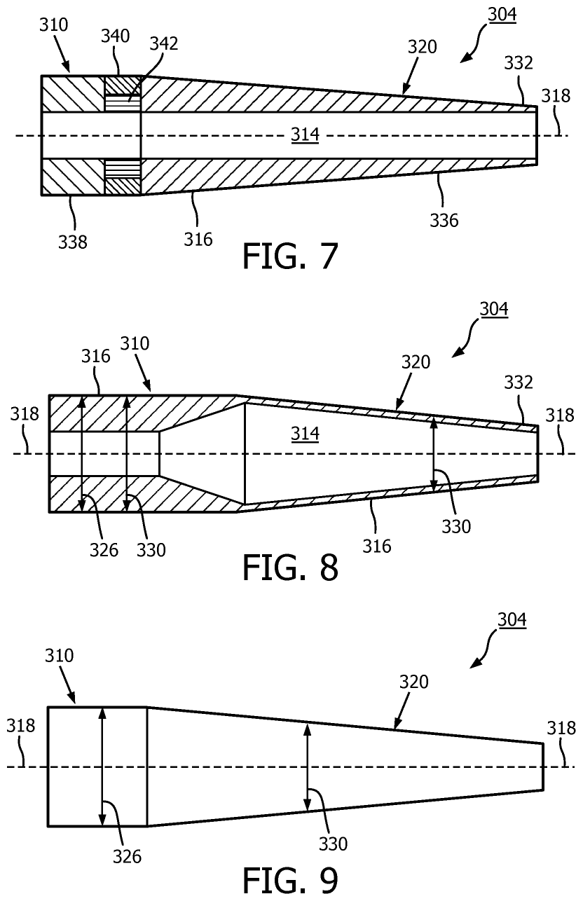

FIG. 7 presents a cross sectional side view of the tip member 304 in which the tip member 304 is made using an injection molding process. This process may be implemented to control the flexibility of the tip member 304. The process includes molding the distal portion 320 using a flexible first material 336 and molding the proximal portion 310 using a second material 338, which is less flexible than the first material 336. This configuration provides a more flexible distal portion 320 of the tip member 304, which is useful for navigating obstructions encountered along the path of the intraluminal device 300. Additionally, this configuration provides an optimized transition to a less flexible proximal portion 310 of the tip member 304, which is connected to the rigid imaging assembly 110. The first material 336 may be selected from any number of materials with flexible properties including, but not limited to, plastic, polymer, elastomer, polyether block amide, Pebax® (e.g., Pebax® 5533), and/or other suitable materials. Further, the second material 338 may be selected from any number of materials that are less flexible than selected first material 336. The process may be configured to control the quantity of the first material 336 and second material 338 injected into the distal portion 320 and the proximal portion 310 respectively ultimately determining the flexibility of the tip member 304. For example, although FIG. 7 shows a greater quantity of the first material 336 in the tip member 304, depending on the desired magnitude of rigidity of the tip member 304 the injection molding process may be modified to increase the quantity of the second material 338 in the proximal portion 310.

The tip member 304 in FIG. 7 contains features that are similar to the tip member 304 presented in FIG. 5c, but also includes a transition region 340 formed from both the first material 336 and the second material 338 and disposed between the proximal portion 310 and the distal portion 320. The transition region 340 includes an interlocking assembly 342, which functions to create a bond between the first material 336 and the second material 338. The interlocking assembly 342 may employ any number of methods or apparatuses for securing the first material 336 and the second material 338 such as, but not limited to a ribbed or textured interface region. Although, FIG. 7 describes two materials being used in the injection molding process to form the tip member 304, it will be appreciated that molding process may employ any number of materials with differing magnitudes of flexibility.

FIG. 8 illustrates a cross sectional side view of the tip member 304 in which the proximal portion 310 has a constant diameter 330 while the wall 316 thickness of the tip member 304 varies along the longitudinal axis 318 and the distal portion 320 has a varying diameter 330 while wall 316 thickness of the tip member 304 is constant along the longitudinal axis 318. Similar to the tip member 304 described with respect to FIG. 7, the tip member 304 presented in FIG. 8 contains features that are similar to the tip member 304 presented in FIG. 5c except for the geometry of the lumen 314. It will be appreciated that shape of the lumen 314 may derive from any number of linear or non-linear geometries as desired. The tip member 304 presented in FIG. 8, illustrates an alternative approach to controlling the flexibility of the tip member 304 with the use of one material as opposed to multiple (e.g. a first material 336 and a second material 338 as discussed with reference to FIG. 7). By increasing the wall 316 thickness along the proximal portion 310 and decreasing the wall 316 thickness along the distal portion 320 around the lumen 314, the tip member 304 may be configured to contain flexibility at the distal portion 320 and less flexibility at the proximal portion 310.

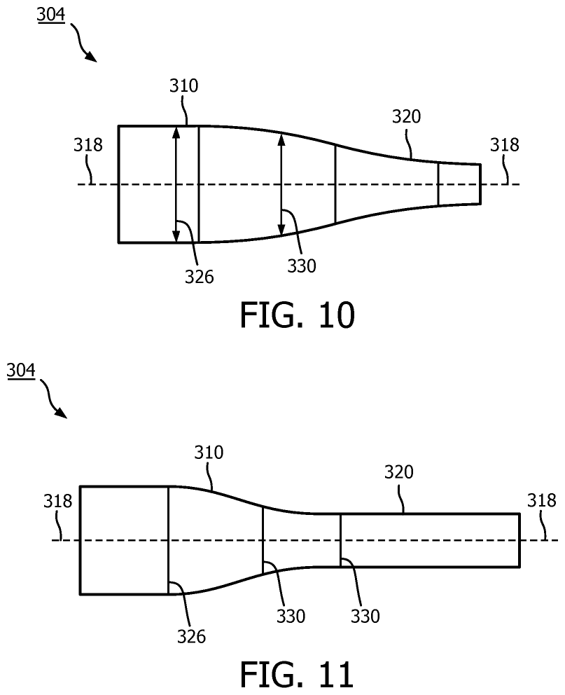

FIGS. 9, 10, and 11 present various types of tip member 304 crossing profiles containing different geometries, which may be situationally used to facilitate translation through or around difficult anatomies. In FIG. 9, a side view of a tip member 304 with a ramp type crossing profile is presented. The ramp type crossing profile has a small outer diameter 330 at the distal portion 320, which gradually increases on a linear slope with respect to the longitudinal axis 318 until reaching the proximal portion 310. The proximal portion 310 may include a profile segment with slope of zero. The use of a tip member 304 with a ramp type crossing profile is advantageous in situations where such as traversing a tight bend within vasculature or other body lumen, where a thin and flexible leading edge consistently transitions to a thicker, less flexible proximal edge. In FIG. 10, a side view of the tip member 304 with a slope type crossing profile is presented. Similar to the ramp type crossing profile, the slope type crossing profile also has a smaller outer diameter 330 at the distal portion 320, which gradually increases along the longitudinal axis 318 towards the proximal portion 310. However, in lieu of increasing linearly, the outer diameter 330 increases along a curvilinear slope from the distal portion 320 to the proximal portion 310. The use of a tip member 304 with a slope type crossing profile is advantageous in situations where such as crossing a partial or complete occlusion within vasculature or other body lumen where the tip ramp would act as a wedge. In FIG. 11, a side view of the tip member 304 with step type crossing profile is presented. Similar to the ramp and slope type crossing profiles of FIGS. 9 and 10, the step type crossing profile has a smaller diameter 330 in the distal portion 320 than in the proximal portion 310. However, in the step type crossing profile, the smaller diameter 330 is maintained throughout the distal portion 320 on a slope of zero until encountering the proximal portion 310 where it increases along a curvilinear slope to a larger diameter 330. The use of a tip member 304 with a step type crossing profile is advantageous in situations where such as crossing a stent within vasculature or other body lumen where a flexible distal portion is desirable to avoid pushing the guide wire and leading edge of the tip against the stent struts. It will be appreciated that the lengths of each distal portion 320 and proximal portion 310 of the profiles in each tip member 304 as well as their respective slopes and radii may be optimized for general use or for specific clinical scenarios.

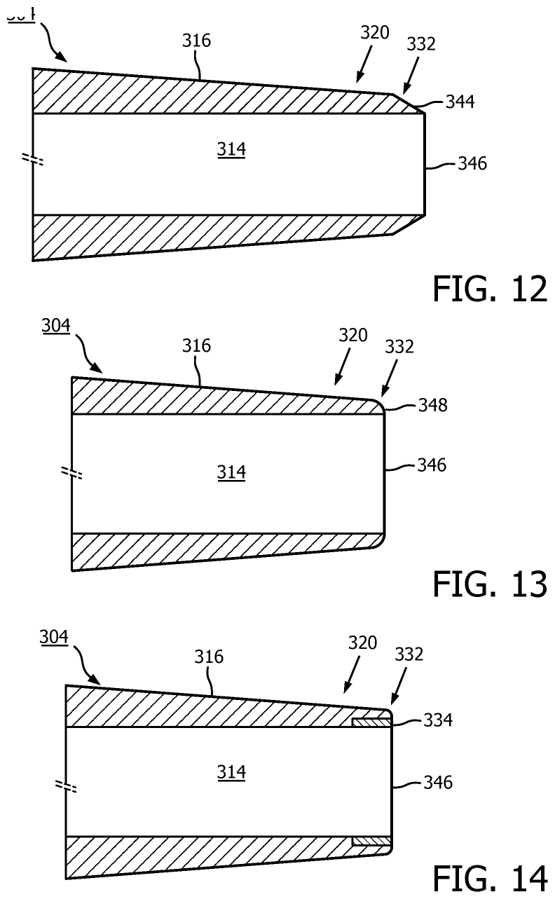

FIGS. 12, 13, and 14 present various types of distal ends 332 of the tip member 304, with profiles containing different geometries, which may be situationally used to prevent deformation of the tip member 304 upon encountering an obstruction. The tip member 304 may be given a first color and the distal end 332 may be given a second color to assist in the guide wire 118 loading process. As previously discussed, the distal end 332, is disposed at the most distal position of the distal portion 320. In FIG. 12 a cross sectional side view of a tip member 304 with a bevel distal end is presented. The distal end 320 contains an outer diameter 344 that linearly slopes away from the wall 316 of the tip member 304 towards an edge 346 of distal end 332. The use of a tip member 304 with a bevel distal end 332 is advantageous in situations where the device is traversing geometry within vasculature or other body lumen that could catch on the tip (e.g. an occlusion or stent). In FIG. 13 a cross sectional the side view of a tip member 304 with a radial distal end 332 is presented. The distal end 332 contains an outer diameter 348 that slopes away from the wall 316 of the tip member 304 in a curvilinear manner towards the edge 346 of the distal end 332. The use of a tip member 304 with a radial distal end 332 is advantageous in situations where the device is traversing a bend within vasculature or other body lumen (especially while on a stiff segment of guide wire) where additional material thickness is needed to prevent tip material deformation. In FIG. 14, a cross sectional side view of the tip member 304 with a reinforcing apparatus 334 is presented. The reinforcing apparatus 334 may be disposed about an outer diameter 350 of the lumen at the edge 346 of the distal end 332. The reinforcing apparatus 334 may also be given a second color to distinguish it from the tip member 304. It will be appreciated that the reinforcing apparatus 334 may be used with any distal end 332 geometrical profile.

1. An intraluminal imaging device, comprising:
a flexible elongate member configured to be inserted into a lumen of a patient, the flexible elongate member comprising a proximal portion and a distal portion;
an ultrasound imaging assembly disposed at the distal portion and configured to obtain ultrasound imaging data while positioned within the lumen of the patient; and
a tip member disposed at the distal portion of the flexible elongate member, the tip member comprising a cavity adjacent to the ultrasound imaging assembly and configured to be filled with an adhesive to couple the tip member and the ultrasound imaging assembly.

2. The device of claim 1, wherein the cavity comprises a junction region at a proximal portion of the tip member, and the cavity comprises a smaller outer diameter relative to the proximal portion of the tip member.

3. The device of claim 2, wherein the cavity comprises a linear outer diameter.

4. The device of claim 2, wherein the cavity further comprises a sloped outer diameter.

5. The device of claim 2, wherein a distal portion of the tip member comprises a crossing region configured to cross an occlusion of the lumen, wherein an outer diameter of the crossing region decreases along a longitudinal axis of the flexible elongate member.

6. The device of claim 5, wherein the crossing region of the tip member comprises a linear outer diameter.

7. The device of claim 5, wherein the crossing region of the tip member comprises a curvilinear outer diameter.

8. The device of claim 5, wherein a distal end of the tip member is shaped to facilitate crossing the occlusion.

9. The device of claim 8, wherein the distal end of the tip member comprises a linear outer diameter.

10. The device of claim 8, wherein the distal end of the tip member comprises a curvilinear outer diameter.

11. The device of claim 8, wherein the distal end of the tip member comprises a reinforcing apparatus.

12. The device of claim 11, wherein the reinforcing apparatus comprises a first color and the tip member comprises a second color different than the first color.

13. The device of claim 2, wherein the proximal portion of the tip member comprises a first material and the distal portion of the tip member comprises a second material.

14. The device of claim 2, wherein the tip member comprises an inner diameter associated with a lumen extending therethrough, wherein the inner diameter comprises an engagement feature configured to contact at least portion of the ultrasound imaging assembly disposed within the lumen.

15. An intraluminal imaging device, comprising:
a flexible elongate member configured to be inserted into a lumen of a patient, the flexible elongate member comprising a proximal portion and a distal portion;
an ultrasound imaging assembly disposed at the distal portion and configured to obtain ultrasound imaging data while positioned within the lumen of the patient; and
a tip member at the distal portion of the flexible elongate member and comprising a first material at a distal portion of the tip member and a second material at a proximal portion of the tip member.

16. The device of claim 15, wherein the first material is less rigid than the second material such that the distal portion of the tip member is more flexible than the proximal portion of the tip member.

17. The device of claim 15, further comprising a transition region between the proximal portion and the distal portion, the transition region comprised of the first material and the second material.

18. An intraluminal imaging device, comprising:
a flexible elongate member configured to be inserted into a lumen of a patient, the flexible elongate member comprising a proximal portion and a distal portion;
an ultrasound imaging assembly disposed at the distal portion and configured to obtain ultrasound imaging data while positioned within the lumen of the patient; and
a tip member at the distal portion of the flexible elongate member and comprising a proximal portion and a distal portion, wherein the proximal portion of the tip member comprises linear outer diameter and varying wall thickness, and the distal portion of the tip member comprises a varying outer diameter and a constant wall thickness.

19. The device of claim 18, wherein the wall thickness of the proximal portion of the tip member in is greater than the wall thickness of the distal portion of the tip member.
